(19) United States
(12) Patent Application Publication
(10) Pub. No.: US 2026/0020894 A1
(43) Pub. Date: Jan. 22, 2026

(54) MEDICAL ROBOT SYSTEM

(71) Applicant: Erbe Elektromedizin GmbH, Tuebingen (DE)

(72) Inventor: Peter SELIG, Nehren (DE)

(21) Appl. No.: 19/268,644

(22) Filed: Jul. 14, 2025

(30) Foreign Application Priority Data
Jul. 22, 2024 (EP) 24190034.9

(51) Int. Cl. A61B 18/12; A61B 18/00; A61B 18/04; A61B 18/16; A61B 34/35; B25J 15/04

(52) U.S. Cl. CPC A61B 18/1206; A61B 18/042; A61B 18/16; A61B 34/35; A61B 34/37; B25J 15/0408; B25J 15/0483; A61B 2018/00589; A61B 2018/00601; A61B 2018/00642; A61B 2018/00732; A61B 2018/00827; A61B 2018/1266; A61B 2018/162

(57) ABSTRACT

The disclosure refers to a medical robot system having a robot comprising at least one robot arm. On the at least one robot arm at least one instrument unit is held and can be moved and/or positioned by means of robot arm. Each instrument unit has a medical instrument, preferably electrosurgical instrument, as well as a generator that creates and provides an output signal (A) for the instrument, particularly an (RF) output signal. On the robot arm an interface is arranged with which each instrument unit held on the robot arm can be electrically and/or optically coupled for establishing a communication connection. This connection can be wired and/or wireless. The robot or robot arm has at least one generator control unit, wherein each interface is communicatively connected with an assigned generator control unit, in order to transmit a generator control signal (C) from generator control unit to the at least one instrument unit connected to the interface. Thus generator and generator control unit are separated from one another, wherein generator is located outside robot, while the at least one generator control unit forms part of robot and can be integrated in robot or robot arm.

### CROSS-REFERENCE TO RELATED APPLICATION

This application claims priority to European Patent Application No. 24190034.9, filed Jul. 22, 2024, the entirety of which is incorporated herein.

### TECHNICAL FIELD

The invention refers to a medical robot system. By means of the robot system an operator, for example a surgeon, can treat a human or animal patient. The robot system comprises a robot having at least one robot arm. Each provided robot arm can hold a medical instrument or also multiple medical instruments. By means of the at least one robot arm a used medical instrument can be positioned and moved, for example during treatment of biological tissue of the patient.

### BACKGROUND

A robot for RF surgery is known from DE 10 2013 002 832 A1. The robot has at least one robot arm and a mechanical interface for releasably attaching a surgical instrument. Additionally, an RF interface for transmission of an RF current to the surgical instrument is provided. Thus, an additional electric line connection from the instrument to an RF generator outside the robot arm can be avoided. The RF generator is integrated in the robot arm and is controlled by means of a control device of the robot.

US 2016/0270840 A1 refers to an electrosurgical instrument in which the generator is integrated inside the instrument housing.

A modular device for robot-supported electrical surgery is known from EP 4 192 379 A1. An instrument holder is provided on a robot arm to connect a medical instrument with the robot arm. Cavities are present in the instrument holder in which electrosurgical capsules can be inserted. Inside the capsule a control module, a generator module and an output module are present. The output module is electrically connected to an output connection. Via the output connection electromagnetic signals can be transmitted to the instrument that is electrically connected with the output connection.

A robot system is described in EP 3 852 664 A1 in which a generator for a medical instrument is arranged on a robot arm.

Starting from the prior art it can be considered as object of the present invention to provide an improved robot system that allows high electromagnetic compatibility on one hand and a flexible and simple use of different medical instruments on the other hand.

This object is solved, for example, by means of a medical robot system having the features of claim 1.

The medical robot system according to the present disclosure comprises a robot having at least one robot arm. The robot arm is configured to hold one or multiple instrument units. For this purpose, the robot arm can have a holding device for one or for more instrument units. By means of the holding device a releasable connection is established with the instrument unit, for example a force-fit and/or form-fit connection.

Each instrument unit comprises a medical instrument, for example an electrosurgical instrument. The instrument can be a monopolar or bipolar instrument. The instrument can be configured for the open surgical or laparoscopic use. The medical instrument can also be an endoscopic instrument and can be used in combination with an endoscope.

Preferably the instrument is an electrosurgical instrument. The instrument can comprise one or more instrument electrodes. For example, two provided instrument electrodes can be used as coagulation electrodes. In addition or as an alternative, the instrument can comprise a cutting electrode. The provided instrument electrodes can also have different functions depending on the requested operating condition of the instrument. For example, a coagulation electrode can be used as a neutral electrode during cutting with a cutting electrode.

In addition, each instrument unit includes a generator. The generator is electrically connected with the medical instrument. The generator can be part of the instrument and can be arranged in the instrument housing, for example. The generator can also be a separate component that is mechanically releasably or unreleasably connected with the medical instrument. In this case the generator and the instrument are electrically and mechanically connected with one another, wherein the connection can be a plug connection, a latched connection or another form-fit and/or force-fit connection, for example.

The generator is configured to provide an electrical output signal alternating with a frequency for the assigned instrument of the instrument unit. The output signal can be an alternating voltage and/or an alternating current. The output signal is particularly a high-frequency signal having a frequency in a range of 100 kHz up to 5 MHz inclusively and preferably from 300 kHz up to 600 KHz inclusively. By means of the generator additional parameters can be adjusted, for example a crest factor of the output signal and/or a waveform of the output signal and/or at least one modulation parameter of a modulated output signal (for example duty cycle of a pulse width modulated output signal).

The robot has a generator control unit. Also multiple generator control units can be provided. Each generator control unit can be assigned to a single robot arm or to multiple robot arms. If a robot arm can hold multiple instrument units, also multiple generator control units can be provided for this robot arm as an option.

Each generator control unit is configured to control at least one or exactly one assigned generator. For this purpose, the generator control unit is connected with an interface arranged on the robot arm. The interface is preferably located in proximity to the instrument holding device for the at least one instrument unit. The connection between the generator control unit and the interface can be realized by means of an electrical line that is arranged inside or on the robot arm. The interface comprises a control connection or multiple control connections, wherein respectively one generator of an instrument unit can be connected to each control connection. If the electrical connection between the generator and the control connection is established, a generator control signal created by the generator control unit can be transmitted to the connected generator.

Due to the separation between the at least one generator control unit of the robot and the at least one generator, a higher flexibility during use of different instruments is achieved. Each instrument unit has its own generator that is configured for the assigned instrument (for example the electrosurgical instrument) of this instrument unit. The generator can be held indirectly by arranging the instrument on the robot arm in a simple manner. Due to the arrangement of the generator directly adjacent to the instrument for which it provides the electrical output signal, electromagnetic interferences can be kept low, which in turn contributes to a very good electromagnetic compatibility (EMC) of the robot system. The output signal, which is particularly a high-frequency output signal, has to be transmitted only over a short distance (that means a short length of the electrical line) from the generator to the instrument or to the at least one instrument electrode of the instrument. If electromagnetic shieldings are necessary, the efforts for this purpose are low.

Depending on the application, one or more required instrument units can be very simply connected to the generator control unit of the robot via the interface on the robot arm. An exchange of robot components is not required.

A single generator control unit of the robot is sufficient in general. In each case the number of instrument units and thus the number of generators can be larger than the number of provided generator control units. In doing so, the control technology related number of components can be low.

During use of a bipolar instrument in the robot system a current is guided from an electrode into the tissue to be treated and from there via an additional instrument electrode back to the generator. If the instrument is a monopolar instrument, a current introduced by means of an instrument electrode into the biological tissue is guided back to the generator via a neutral electrode attached to the patient. For this purpose, a neutral electrode connection for a neutral electrode can be present on the generator and/or the interface of the robot arm. The neutral electrode can be electrically connected with multiple generators of different instrument units indirectly via the interface on the robot arm or alternatively directly.

It is advantageous if the interface comprises at least one identification connection. By means of the identification connection, an identification signal can be transmitted to the assigned generator control unit. The identification signal allows an identification of an instrument or an instrument unit arranged on this robot arm in that it describes or comprises an identifier assigned to the instrument unit, for example. The identifier is preferably unchangeable. The identifier can be stored in a data storage of the instrument unit. Based on the received identification signal, the generator control unit can determine which kind or which type of medical instrument is present on the robot arm and can thereupon initiate an adapted control of the instrument unit via the control connection of the interface. The kind or type of the instrument can be characterized by one characteristic or multiple arbitrary characteristics in the following:
the number of provided electrodes,
the configuration as a monopolar or bipolar instrument,
an instrument that requires and/or uses a fluid (gas and/or liquid) for influencing tissue,
the at least one use of the instrument (cutting, coagulating, fusing, etc.).

The instrument unit can be connected to the identification connection of the interface in wireless or wired manner. The connection can be established in electrical and/or electromagnetic and/or optical manner.

For example, in an embodiment a detection unit can be provided that is connected or can be connected with the identification connection and that is configured to detect an identifier of the assigned instrument unit or the assigned instrument. Based on the identifier the generator control unit can determine which instrument type is involved and how this instrument type has to be controlled.

The detection unit can detect the identifier in wireless or wired manner, wherein the connection can be established electrically, electromagnetically or optically. For example, the detection unit can use a near field communication (NFC) or an RFID-connection (radio-frequency identification) in order to detect the identifier. The detection unit can be arranged in the generator of the instrument unit and/or in the interface of the robot arm. Additionally or alternatively, the identifier can be an optically detectable code, for example a two-dimensional code (bar code, QR code, etc.).

It is additionally advantageous if the interface comprises at least one energy supply connection. The energy supply connection is connected to an energy source of the robot or the robot system, wherein the energy source particularly provides a direct voltage and/or a direct current. Thereby, either the direct voltage or the direct current can be impressed.

It is preferred if the interface comprises at least one feedback connection. Each feedback connection can be connected to one generator of an instrument unit held on this robot arm. Via the feedback connection, a feedback signal can be transmitted to the generator control unit or the generator control unit assigned to this interface.

The feedback signal can characterize a currently present operating parameter of the generator and/or a current use of the instrument. The feedback signal can be an electrical signal, and/or an optical signal or a light signal. For example, the feedback signal can describe the output signal that the generator transmits to the instrument. Also, the feedback signal can characterize the currently treated tissue and/or the current application of the instrument. For example, the feedback signal can characterize a light appearance created upon the creation of a spark at the at least one instrument electrode.

In an embodiment the interface has a fluid connection connected to a fluid source of the robot or the robot system. By means of the fluid source a fluid can be supplied to the fluid connection. The instrument of an assigned instrument unit held on the concerned robot arm can be connected to the fluid connection. In doing so, the instrument can be supplied with fluid, such as a gas (for example a carbon dioxide or argon) or a liquid (for example water). The number of fluid connections and the number of provided fluids can be arbitrarily selected.

### BRIEF DESCRIPTION OF THE DRAWINGS

Advantageous configurations of the invention are derived from the dependent claims, the description and the drawing. In the following preferred embodiments of the invention are explained in detail based on the attached drawing. The drawing shows:

FIG. 1 a schematic basic illustration of an embodiment of a robot system having at least one robot arm that comprises an interface for at least one instrument unit,

FIGS. 2 to 4 show block diagrams of different configurations of a realization of the interface as well as the instrument unit respectively.

### DETAILED DESCRIPTION

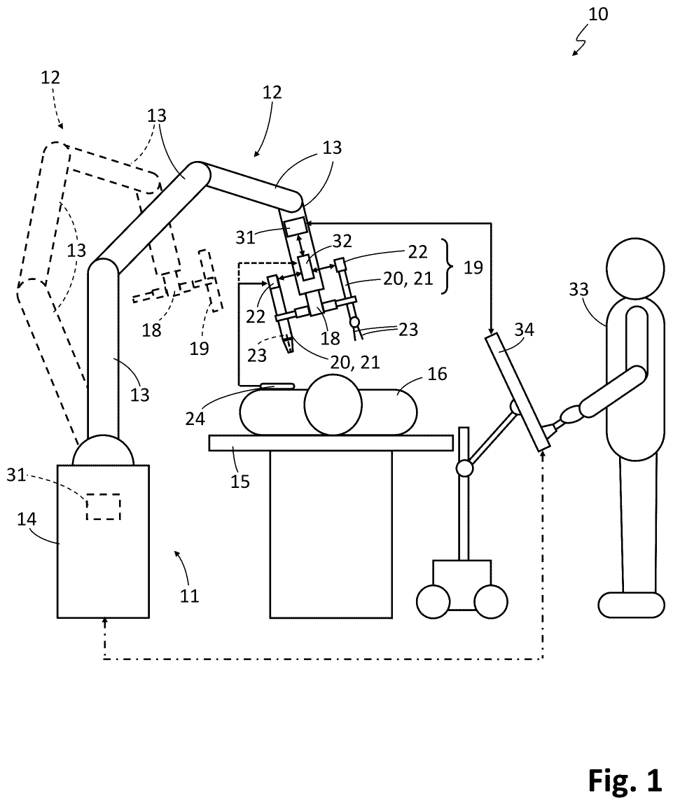

In FIG. 1 an embodiment of the robot system 10 is shown in a schematic basic illustration. The medical robot system 10 has a robot 11 having at least one robot arm 12. Each robot arm 12 has multiple arm elements 13 that are movably connected with each other. The arm elements 13 can be supported on each other in a manner to be pivotable or rotatable around respectively one axis or multiple different axes. The robot 11 has a base 14 on which the at least one robot arm 12 is arranged. By means of base 14 the robot 11 can be positioned in a treatment room or operating room, for example adjacent to a treatment table or operating table 15. Alternatively, the base 14 can also be arranged on the operating table or another mobile or immobile device.

On a free end, which is the end opposite the base 14 of the robot arm 12 according to the example, at least one of the provided robot arms 12 has a holding device 18 for a single instrument unit 19 or for multiple instrument units 19. The holding device 18 is configured for establishment of a form-fit and/or force-fit mechanical connection with the at least one instrument unit 19. The entire holding device 18 can be moved and positioned by means of the concerned robot arm 12.

The holding device 18 can be configured to individually move and/or position the at least one held instrument unit 19, particularly relative to the arm element 13 of robot arm 12 on which the holding device 18 is attached. The holding device 18 can comprise at least one linear axis and/or at least one rotational axis in order to move and position the at least one instrument unit 19.

If holding device 18 is configured to hold multiple instrument units 19, one or more currently not used instrument units 19 can be moved into a rest position in which they do not form an interfering contour for the currently used instrument unit 19.

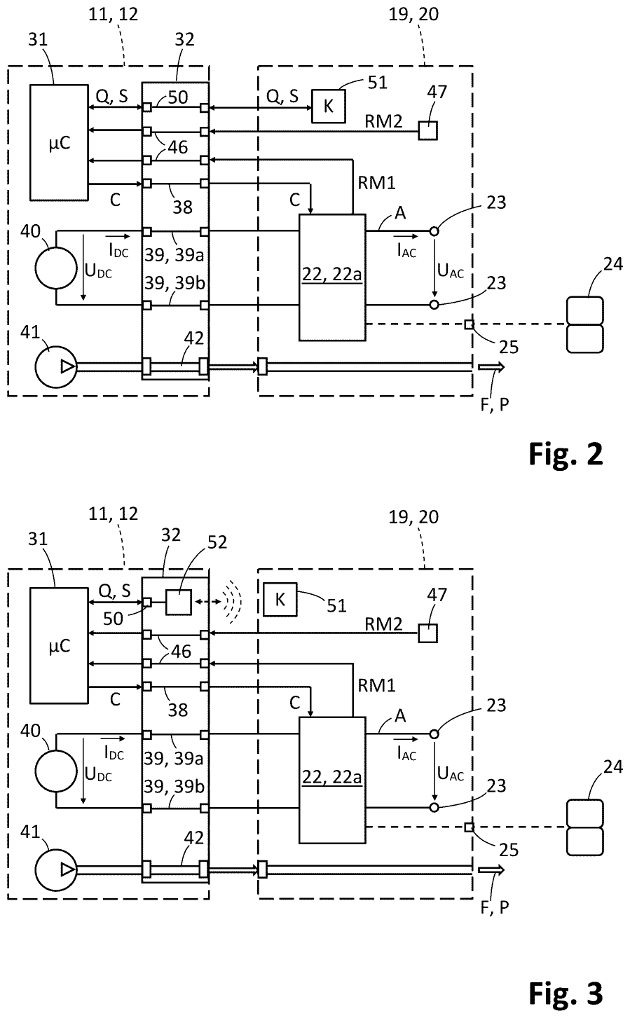

Each instrument unit 19 comprises a medical instrument 20, particularly an electrosurgical instrument 21 as well as a generator 22. Each generator of an instrument unit 19 is configured to provide an output signal A having a predefined frequency for the assigned instrument 20 (FIGS. 2 to 4). For this purpose, generator 22 comprises a converter circuit 22a according to the example. The output signal A can be an alternating voltage U_{AC} and/or an alternating current I_{AC}. Thereby either the alternating voltage U_{AC} or the alternating current I_{AC} can be impressed. The frequency of the output signal A is particularly at least 100 kHz and preferably at most 5 MHz. The frequency can be in a range from 300 kHz to 600 kHz, for example.

The output signal A of generator 22 is provided to the assigned instrument 20 of the same instrument unit 19. According to the example instrument 20 has at least one instrument electrode 23 to which the output signal A is supplied. For example, the alternating voltage U_{AC} can be applied between two instrument electrodes 23. If an electrical circuit is closed via the biological tissue of the patient 16 to be treated an alternating current I_{AC} can flow via one instrument electrode 23 to the treated tissue of patient 16 and via another instrument electrode 23 back to generator 22. This is possible if instrument 20 is configured as bipolar instrument and comprises at least two instrument electrodes 23.

Alternatively, instrument 20 can be configured as monopolar instrument and can have, for example, only one single instrument electrode 23 or multiple instrument electrodes 23 having a common electrical potential. In this case a neutral electrode 24 can be attached to the patient 16 in an electrically conducting manner and can be electrically connected with generator 22 via a neutral electrode connection 25, as optionally depicted in dashed lines in FIGS. 2 to 4. The neutral electrode 24 is only required if in the robot system 10 for the treatment of a patient 16 at least one instrument 20 is used that is configured as monopolar instrument.

Robot 11 comprises at least one generator control unit 31. For each provided robot arm 12 a separate generator control unit 31 can be provided in an embodiment. It is also possible to assign one common generator control unit 31 to multiple or all provided robot arms 12. In another modified embodiment for each instrument unit 19 that can be held by robot 11 respectively one individual generator control unit 31 can be provided.

The generator control unit 31 can be arranged in or on robot arm 12, for example, as it shown in FIG. 1 by way of example. The generator control unit 31 can be arranged in or on an arm element 13, for example in the arm element 13 that supports the holding device 18. Alternatively, the at least one generator control unit 31 can be arranged in another arm element 13 or in the base 14, as it is exemplarily indicated by dashed lines in FIG. 1.

The generator control unit 31 is communicatively connected with an interface 32 of at least one robot arm 12. This connection is preferably an electrical connection, but can additionally or alternatively also be an optical connection. Preferably, the at least one connection between the generator control unit 31 and the interface 32 is realized by means of one electrical and/or optical line.

Each robot arm 12 comprises an interface 32. The interface 32 is preferably arranged adjacent to the holding device 18 and is located in or on the arm element 13 on which also the holding device 18 for the at least one instrument unit 19 is arranged, according to the embodiment. By means of interface 32 the at least one instrument unit 19 held on the concerned robot arm 12 can be connected with the generator control unit 31 for this robot arm 12, particularly via an electrical and/or optical connection. Basically, the electrical and/or optical connection can be wireless and/or wired.

As shown in FIG. 1, an operator 32 (for example a surgeon) can control robot 11 by means of an operating device 34. For example, the operator 33 can transmit control commands by means of the operating device 34 to the at least one generator control unit in order to control the operation of the at least one instrument unit 19. By means of the operating unit 34 also actuators of robot 11, particularly of the at least one robot arm 12 and the at least one holding device 18, can be controlled in order to move and/or position the at least one medical instrument 20, which is schematically illustrated in FIG. 1 by means of double arrow depicted in dot-dashed manner. The operating device 34 can have the operating elements necessary for this purpose. The operating device 34 can also comprise output means (for example acoustical and/or optical output means) in order to provide information about the current use of robot 11 or the at least one medical instrument 20 to the operator 32. For example, the operating device 34 can comprise a screen, particularly a touch-sensitive screen, as an interface.

The various configuration possibilities of instrument unit 19, generator 22 as well as interface 34 are illustrated based on embodiments in the block diagrams of FIGS. 2 to 4.

The interface 32 of robot arm 12 comprises at least one control connection 38. The control connection 38 is communicatively connected with generator control unit 31, so that a generator control signal C can be transmitted to the control connection 38 and from there further to a generator 22 of an instrument unit 19 connected to the control connection 38. Thus, operation of generator 22 is controlled by means of generator control unit 31.

The high-frequency output signal A is thus created outside of robot arm 12 in the instrument unit 19 using generator 22 and is provided to the instrument 20 of the respective instrument unit 19 (for example high-frequency alternating current I_{AC} and/or high-frequency alternating voltage U_{AC}). By means of generator control signal C the parameters for the output signal A can be predefined and adjusted, for example a frequency and/or an amplitude and/or a crest factor and/or a waveform and/or a duty cycle in case of a pulse width modulated signal, etc. In doing so, the operation of instrument 20 can be controlled in an appropriate manner, for example for coagulation, fusion or cutting of biological tissue of a specific tissue type.

In order for generator 22 to create the output signal A for instrument 20 the interface can have an energy supply connection 39. In the embodiment interface 32 has a first energy supply connection 39a as well as a second energy supply connection 39b. The two poles of an energy source 40 are connected to the two energy supply connections 39a, 39b. The energy source 40 can be a direct voltage source or a direct current source, for example, for providing an impressed direct voltage U_{DC} or an impressed direct current I_{DC}. Via energy supply connections 39a, 39b the electrical energy is transferred to the generator 22 of an instrument unit 19 connected to the energy supply connections 39a, 39b. Using the provided electrical energy generator 22 (or converter circuit 22a of generator 22) can create the output signal A.

Thus, interface 32 can also serve for energy or power supply of generator 22. Alternatively to this, instrument unit 19 can also be supplied with electrical energy without interposition of interface 32.

Optionally interface 32 can comprise a fluid connection 42 that is fluidically connected or can be fluidically connected with a fluid source 41. By means of fluid connection 42, an instrument 20 of an instrument unit 19 connected thereto can be supplied with at least one fluid, for example a gas and/or a liquid, if necessary or advantageous for the operation of the concerned instrument 20. For example, by means of fluid connection 42 of interface 32 an instrument 20 configured as plasma instrument can be supplied with a suitable gas (for example argon). By means of the at least one instrument electrode 23 a plasma can be ignited or created and can be ejected from instrument 20 as plasma stream P. Also other fluids, for example water or another liquid, can be supplied to instrument 20 in order to create and eject a respective fluid stream F.

In the embodiments illustrated in FIGS. 2 to 4, interface 32 comprises at least one feedback connection 46. The at least one feedback connection 46 is communicatively connected with the concerned generator control unit 31. Via each feedback connection 46, a feedback signal RMi (i=1, 2, . . . , n) can be transmitted from an instrument unit 19 connected thereto. Only by way of example, respectively two feedback connections 46 are illustrated in FIGS. 2 to 4 for transmission of a first feedback signal RM1 and a second feedback signal RM2. The at least one feedback signal RMi can be an electrical and/or optical signal.

The first feedback signal RM1 can characterize, for example, a currently present operating parameter of generator 22, for example an alternating current I_{AC} that results from an impressed alternating voltage U_{AC} used as output signal A or vice versa. For example, second feedback signal RM2 can characterize a spark creation at the at least one instrument electrode 23, which has been detected by means of a suitable sensor 47.

In the embodiments according to the invention, as an option, an automatic recognition of an instrument 20 or an instrument type can be realized. In doing so, for example, it can be automatically determined in generator control unit 31 which instrument type is used in an instrument unit 19 connected to interface 32. For example, instrument types can be distinguished from one another, such as plasma instruments (for example plasma coagulation instruments), electrosurgical cutting instruments, electrosurgical coagulation instruments having two or more coagulation electrodes, thermo-fusion instruments, etc. Additionally, it can also be determined whether the concerned instrument 20 is a monopolar or bipolar instrument. In general, the kind or the type of an instrument 20 can be characterized by one or more of the characteristics listed in the following in any arbitrary combination:
the number of provided electrodes,
the configuration as monopolar or bipolar instrument,
an instrument that requires and/or uses a fluid (gas and/or liquid),
the at least one use of the instrument (cutting, coagulating, fusing, etc.).

For identification of an instrument 20 an identifier K can be transmitted via interface 32 to the generator control unit 31. For this purpose, interface 32 comprises in the embodiment an identification connection 50 by means of which an identification signal S can be transmitted to generator control unit 31. For example, the identifier K can be read from a data carrier 51 of instrument unit 19 in wired or wireless manner by generator control unit 31 and can be transmitted as identification signal S to generator control unit 31. The data carrier 51 can be part of the generator 22 or the instrument 20.

Reading of the identifier K can be initiated by generator control unit 31, for example in that generator control unit 31 transmits a request signal Q or reading signal to data carrier 51 (FIG. 2). In the embodiment illustrated in FIG. 2 the connection between the identification connection 50 and data carrier 51 is realized as wired connection.

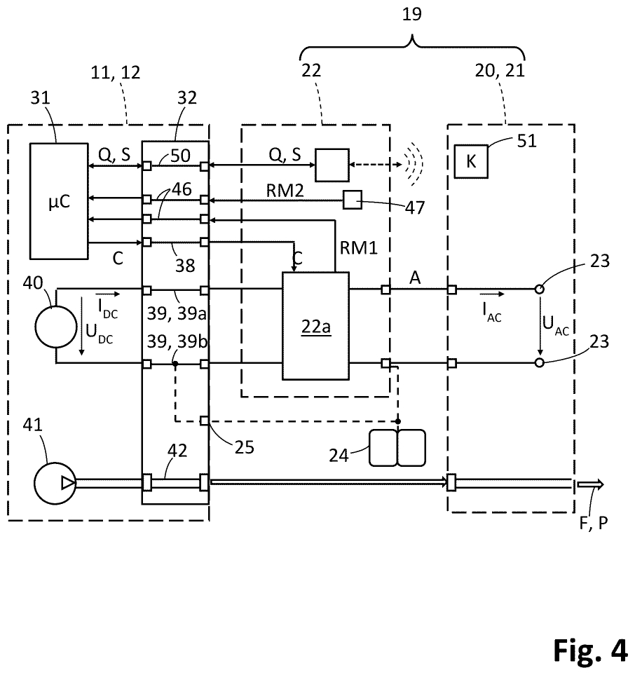

Reading of identifier K from a suitable data carrier 52 can also be carried out by means of a detection unit 52 in modification to the embodiment according to FIG. 2, wherein the detection unit 52 is particularly arranged outside instrument 20, for example in the robot arm 12 or in the interface 32 of robot arm 12 (FIG. 3) or is a component of generator 22 (FIG. 4). For example, detection unit 52 can read the identifier K by means of near field communication (NFC) or by means of an RFID connection from data carrier 52 and can transmit an identification signal S characterizing the identifier K to generator control unit 31. The reading of identifier K by means of detection unit 52 can be triggered by request signal Q that is transmitted from generator control unit 31 to the detection unit 52 via identification connection 50 (FIGS. 3 and 4).

In the embodiments schematically depicted in FIGS. 2 and 3 generator 22 and instrument 20 are configured as common integral components and are particularly arranged in a common housing. In modification to this, generator 22 and instrument 20 could also be realized by separate components that are mechanically connected and are communicatively connected (FIG. 4). For example, generator 22 and instrument 20 could be realized in separate housing parts, wherein the two housing parts are mechanically releasably or unreleasably connected with each other, whereby also a communication connection is established, for example an electrical and/or optical connection.

In the embodiments illustrated in FIGS. 2 to 4 a neutral electrode connection 25 for connecting a neutral electrode 24 to the generator 22 can be provided as an option. Additionally or alternatively, the neutral electrode connection 25 can also be part of interface 32 on robot arm 12 (dashed arrow in FIG. 1). For example, if instrument 20 is configured as monopolar instrument so that one single energy supply connection 39 (for example first energy supply connection 39a) is sufficient for the at least one instrument electrode 23, another energy supply connection (for example second energy supply connection 39b) can be used as neutral electrode connection 25. This possibility is also schematically illustrated in dashed lines in FIG. 4.

The invention refers to a medical robot system 10 having a robot 11 comprising at least one robot arm 12. On the at least one robot arm 12 at least one instrument unit 19 is held and can be moved and/or positioned by means of robot arm 12. Each instrument unit 19 has a medical instrument 20, preferably electrosurgical instrument 21, as well as a generator 22 that creates and provides an output signal A for the instrument 20, particularly an RF output signal. On the robot arm 12 an interface 32 is arranged with which each instrument unit 19 held on the robot arm 12 can be electrically and/or optically coupled for establishing a communication connection. This connection can be wired and/or wireless. The robot 11 or robot arm 12 has at least one generator control unit 31, wherein each interface 32 is communicatively connected with an assigned generator control unit 31, in order to transmit a generator control signal C from generator control unit 31 to the at least one instrument unit 19 connected to the interface 32. Thus generator 22 and generator control unit 31 are separated from one another, wherein generator 22 is located outside robot 11, while the at least one generator control unit 31 forms part of robot 11 and can be integrated in robot 11 or robot arm 12.

1. A medical robot system comprising:
at least one instrument unit respectively comprising a medical instrument, particularly an electrosurgical instrument, and a generator assigned to this instrument, wherein the generator is connected with the instrument and is configured to provide an electrical output signal alternating with a frequency for the assigned instrument,
a robot having at least one generator control unit and having at least one robot arm that is configured to hold the instrument unit or at least one of the provided instrument units,
an interface arranged on the robot arm, wherein the interface is connected with the generator control unit assigned to the robot arm and comprises at least one control connection to which one generator of an instrument unit held at the robot arm can be connected respectively in order to transmit a generator control signal from the generator control unit to the generator.

2. The medical robot system according to claim 1, wherein the generator is releasably or unreleasably attached to the instrument in the instrument unit or in at least one of the provided instrument units.

3. The medical robot system according to claim 1, wherein the generator is arranged inside the instrument of the instrument unit or of at least one of the provided instrument units.

4. The medical robot system according to claim 1, wherein the instrument comprises at least one instrument electrode that is electrically connected with the generator of the instrument unit.

5. The medical robot system according to claim 1, wherein the generator of the instrument unit or of at least one of the provided instrument units and/or the interface comprises a neutral electrode connection for a neutral electrode.

6. The medical robot system according to claim 1, wherein the interface comprises at least one identification connection to which one generator of an instrument unit held at the robot arm can be connected respectively in order to transmit an identification signal(S) to the assigned generator control unit.

7. The medical robot system according to claim 6, wherein the generator and/or the interface comprises a detection unit that can be connected with the identification connection and that is configured to detect an identifier (K) of the assigned instrument.

8. The medical robot system according to claim 1, wherein the interface comprises at least one energy supply connection that is connected with an energy source and to which one generator of an instrument unit held at the robot arm can be connected respectively in order to supply the generator with electrical energy.

9. The medical robot system according to claim 8, wherein a direct voltage (U_{DC}) and/or a direct current (I_{DC}) is provided at the energy supply connection by means of energy source.

10. The medical robot system according to claim 1, wherein the interface comprises at least one feedback connection to which one generator of an instrument unit held at the robot arm can be connected respectively in order to transmit at least one feedback signal (RMi) to the assigned generator control unit.

11. The medical robot system according to claim 10, wherein the at least one feedback signal (RMi) characterizes a currently present operating parameter of the generator and/or a current use of the instrument.

12. The medical robot system according to claim 1, wherein the interface comprises at least one fluid connection that is connected with a fluid source and to which an instrument of an instrument unit held at the robot arm can be connected respectively in order to supply the instrument with the fluid.
